(19) United States
(12) Patent Application Publication
(10) Pub. No.: US 2003/0114805 A1
(43) Pub. Date: Jun. 19, 2003

(54) Absorbent pad for receiving urine and feces and washing method therefor

(76) Inventors: Francine Rainville-Lonn, Beaconsfield (CA); Harvey Lonn, Beaconsfield (CA)

Correspondence Address: OGILVY RENAULT, 1981 MCGILL COLLEGE AVENUE, SUITE 1600, MONTREAL, QC, H3A2Y3, CA

(21) Appl. No.: 10/015,773

(22) Filed: Dec. 17, 2001

(51) Int. Cl. A61F 13/20; A61F 13/15

(52) U.S. Cl. 604/358

(57) ABSTRACT

An absorbent pad for receiving therein urine and feces. The absorbent pad comprises an elongated body having a bottom panel with a peripheral wall projecting from a periphery of the bottom panel to define a concavity, and a layer of absorbent material in the concavity. The bottom panel and the peripheral wall have impermeable outer surfaces such that liquid received in the concavity is retained therein and is absorbed by the layer of absorbent material. The absorbent pad is adapted for being positioned between legs of a person such that the concavity faces a crotch of the person for receiving at least one of urine and feces in the concavity. The peripheral wall is elevated so as to prevent the absorbent layer in the concavity of the absorbent pad from coming into contact with skin of the person.

### TECHNICAL FIELD

The present invention generally relates to an absorbent pad for people having bowel or bladder deficiencies, such as incontinence, and a washing method therefor.

### BACKGROUND ART

There are plural absorbent articles having has a main purpose to absorb urine and other bodily fluids, and retain feces, in order to keep a wearer's clothes dry. Sanitary pads, diapers and other such articles have substantially improved in quality over the last decades with the advent of plastified materials and improved absorbent textiles. Therefore, while the main purpose of the absorbent articles remains the absorption of urine and the retention of feces to keep the clothes dry, these absorbent articles have evolved in order to increase the comfort of the user.

For one thing, the new materials have allowed the absorbent articles to be lighter and smaller. Also, manufacturing methods and processes have been developed to enable the manufacturing of ergonomically shaped absorbent articles. The absorbent articles have also been improved in keeping the wearer dry. Although the absorption rates of the absorbent textiles have increased, an absorbent article often has to receive large quantities of liquids in a short span of time.

Another issue resides in the fact that the absorbent articles must remain in position with respect to the wearer. Therefore, the existing various absorbent articles are configured for being secured to the wearer. For instance, diapers replace the undergarments, thereby ensuring that they stay in position. Some sanitary pads have developed wings, which fold in order to embrace the bottommost portion of the undergarment. However, both of these securing solutions increase the size of the absorbent article, which goes against the idea of minimizing the size and weight of the article for the user's comfort. Also, the wings are not adaptable to the new boxer types of undergarment. U.S. Pat. No. 5,207,662, issued to James on May 4, 1993, U.S. Pat. No. 5,360,422, issued to Brownlee et al. on Nov. 1, 1994, U.S. Pat. No. 6,126,648, issued to Keck et al. on Oct. 3, 2000, and U.S. Pat. No. 6,133,501, issued to Hallock et al. on Oct. 17, 2000, each describe various absorbent pads to be used as part of or adapted to be mounted to a diaper-like garment. U.S. Pat. No. 6,066,121, issued to Lindquist et al. on May 23, 2000, U.S. Pat. No. 5,752,947, issued to Awolin on May 19, 1998, and U.S. Pat. No. 6,241,714 B1, issued to Raidel et al. on Jun. 5, 2001, each describe the pad type of absorbent article which is securable to the undergarment.

Although the absorbent articles are adapted for retaining feces and urine, there is also a need for absorbent articles which are readily washable, such that a person wearing a soiled absorbent article may simply go to nearby facilities, for instance public restrooms, to wash out the soiled article. It is often preferable to perform the washing rapidly to avoid giving off odors, whereby there is a need for a readily achieved method of washing such absorbent articles.

### SUMMARY OF INVENTION

Therefore, it is a feature of the present invention to provide an absorbent pad which substantially overcomes the disadvantages of the prior art and meets the above mentioned need.

It is a further feature of the present invention to provide a method for washing such an absorbent pad for improving the comfort of the wearer.

According to the above features of the present invention, from a broad aspect, there is provided an absorbent pad for receiving therein at least one of urine and feces. The absorbent pad comprises an elongated body having a bottom panel with a peripheral wall projecting from a periphery of the bottom panel to define a concavity, and a layer of absorbent material in the concavity. The bottom panel and the peripheral wall have impermeable outer surfaces such that liquid received in the concavity is retained therein and is absorbed by the layer of absorbent material. The absorbent pad is adapted for being positioned between legs of a person such that the concavity faces a crotch of the person for receiving at least one of urine and feces in the concavity. The peripheral wall is elevated so as to prevent the absorbent layer in the concavity of the absorbent pad from coming into contact with skin of the person.

According to a further broad aspect of the present invention, there is provided a method for washing an absorbent pad having absorbed liquid therein. The method comprises the steps of i) providing handle tabs at opposed ends of the absorbent pad; ii) holding the absorbent pad by the handle tabs; and iii) removing liquid absorbed in the absorbent pad by twisting the tabs in opposed directions to wring the absorbent pad.

According to a still further broad aspect of the present invention, there is provided an absorbent pad in combination with an undergarment. The absorbent pad is adapted for receiving therein at least one of urine and feces. The absorbent pad comprises an elongated body having a bottom panel with a peripheral wall projecting from a periphery of the bottom panel to define a concavity, and a layer of absorbent material in the concavity. The bottom panel and the peripheral wall are impermeable such that liquid received in the concavity is retained therein and is absorbed by the layer of absorbent material. The absorbent pad is adapted for being positioned between legs of a person such that the concavity faces a crotch of the person for receiving at least one of urine and feces in the concavity. An underface of the bottom panel has first fastening members on front and rear ends of the absorbent pad; Second fastening members on the undergarment are provided for corresponding engagement with the first fastening members. The second fastening members are adapted for being fixed to the undergarment so as to secure the absorbent pad to the undergarment.

### BRIEF DESCRIPTION OF DRAWINGS

A preferred embodiment of the present invention will now be described with reference to the accompanying drawings in which:

FIG. 1 is a perspective view of the absorbent pad in accordance of the present invention;

FIG. 2 is a cross-sectional view of the absorbent pad taken along cross-section line 2-2 of FIG. 1;

FIG. 3 is a schematic cross-sectional view of the absorbent pad as positioned with respect to a user's crotch;

FIG. 4 is an exploded view of the absorbent pad in accordance with another embodiment of the present invention; and

FIG. 5 is a bottom plan view of the absorbent pad in accordance with still another embodiment of the present invention.

### DESCRIPTION OF PREFERRED EMBODIMENTS

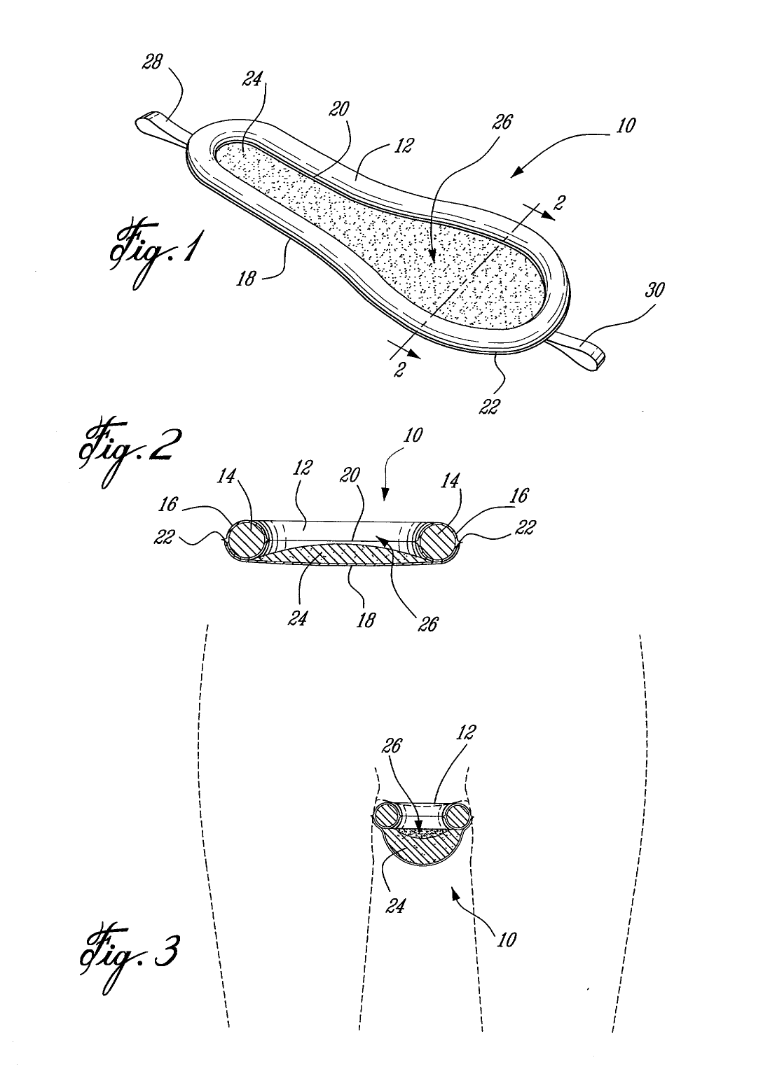

Referring now to the drawings and, more particularly to FIG. 1, the absorbent pad in accordance with the present invention is generally shown at 10. The absorbent pad 10 is shaped like an elongated pear and has a contour rib 12. The contour rib 12, as best seen in FIG. 2, has a generally circular cross-section to provide comfort to the user. The contour rib 12 has a resilient core 14 and an outer plastified layer 16. The resilient core 14 consists preferably of a resilient material, such as a low-density foam. The outer plastified layer 16 is impermeable and, therefore, the resilient core 14 is isolated from liquids which come into contact with the contour rib 12. The outer plastified layer 16 as an exterior surface which is soft to the touch, as it will be in contact with the user's skin.

Still referring to FIG. 2, the contour rib 12 sits on an bottom panel 18, which has a pear-like shaped outer periphery. The bottom panel 18 is sealed with the contour rib 12 at an outer joint line 22 using methods which will be described hereinafter. The assembly of the contour rib 12 to the bottom panel 18 forms a concavity 26. The bottom panel 18 consists of a thin fabric having one of its surfaces plastified. The bottom panel 18 has its plastified surface facing upwards in the concavity 26. The plastified surface renders the bottom panel 18 impermeable, whereas the other surface has a typical fabric finish, and thus has a greater friction coefficient, which allows the absorbent pad 10 to be more stable when positioned in an undergarment. A fabric well suited to be used for the bottom panel is Comfort®.

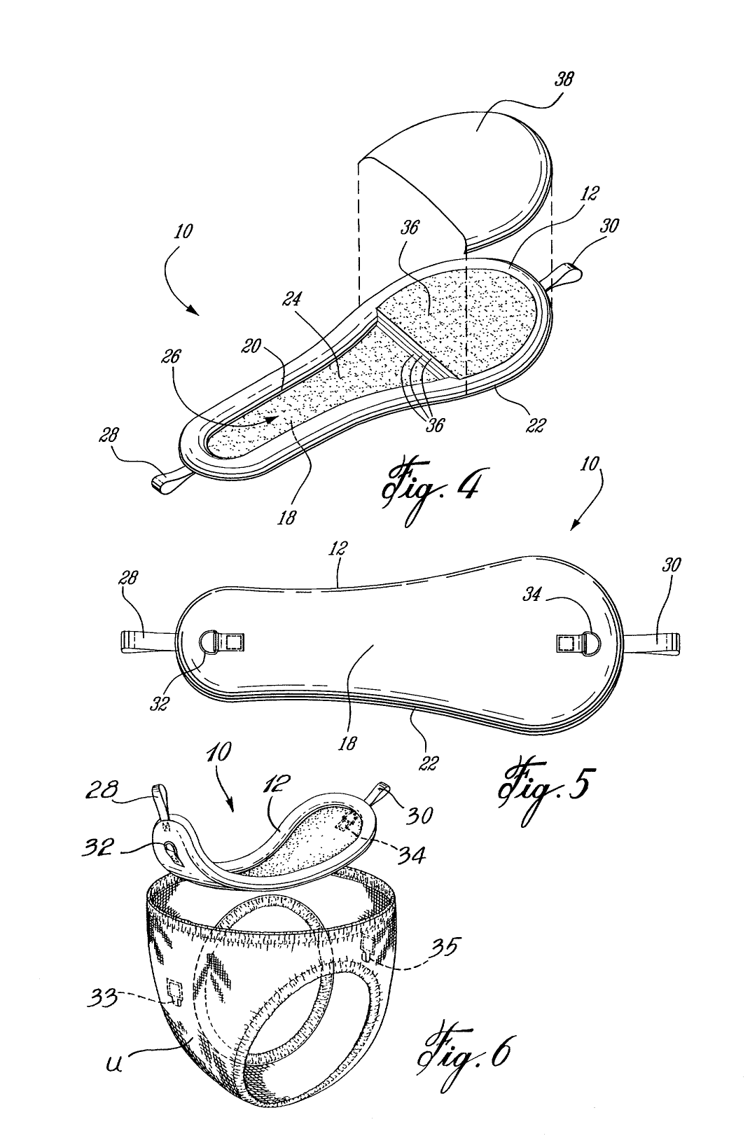

An absorbent layer 24 is received in the concavity 26 and lies on the plastified surface of the bottom panel 18. The periphery of the absorbent layer 24 is sandwiched between the contour rib 12 and the bottom panel 18. This absorbent layer 24 is typically chosen amongst high-absorption materials. A type of polyester well suited for serving as an absorbent layer is the bacteria-free Intera® material. Even though the contour rib 12 is elevated with respect to the absorbent layer 24, it is preferable that the absorbent layer 24 be bacteria-free, as the user's skin may still come into contact therewith. The elongated-pear shape ensures an ergonomic fit of the absorbent pad 10 between the legs of the person when in position in the crotch. The larger end of the absorbent pad 10 is positioned in the buttock area of the user, for retaining feces. Accordingly, a major portion of the concavity 26 is positioned for receiving feces. As seen in FIG. 3, when the absorbent pad 10 is worn, the concavity 26 forms a trough. Therefore, when a liquid is received in the absorbent pad 10, the concavity 26 acts like a dam to hold the liquid therein while it is being absorbed by the absorbent layer 24.

Loops 28 and 30 are positioned at front and rear ends of the absorbent pad 10, respectively. The loops 28 and 30 are secured to the bottom panel 18. The loops 28 and 30 are provided for facilitating the handling of the absorbent pad 10. The absorbent pad 10 can even be wrung with the help of the loops 28 and 30. The absorbent pad 10 is preferably used by people having incontinence problems, which consist in a person's inability to control his/her bowels and/or bladder. Therefore, the absorbent pad 10 may come into use at awkward times. The loops 28 and 30 enable manipulation of the absorbent pad 10 to have the excess feces and/or urine retained therein removed. For instance, a person may remove the absorbent pad 10 in a public restroom to then have the feces and urine rinsed off in a toilet bowl and the absorbed water extracted by the wringing of the absorbent pad 10. The absorbent pad 10 may then be dried off by wiping it with toilet paper. Thereafter, the absorbent pad 10 may be put back into place. The washing steps are achieved with the user only having to manipulate one or both loops 28 and 30, and thus not handling other parts of the absorbent pad 10.

As depicted in FIG. 4, the absorbent pad 10 may be adapted for providing higher absorption in cases where it is used solely for absorbing urine and other bodily liquids. In this case, layers 36 of absorbent material are positioned in the larger end of the concavity 26 of the absorbent pad 10. A cover panel 38 is disposed thereon to cover the layers 36 and act as a barrier between the user's skin and the absorbent material. The cover panel 38 is preferably made of the same material as the bottom panel 18. Therefore, Comfort® is well suited for such a use. The plasticized surface of the cover panel 38 faces downwardly such that the fabric side is in contact with the skin of the user. The cover panel 38 is secured to the contour rib 12, as will be described later. The absorbent material of the layers 36 may be bulkier than the absorbent layer 24, so as to completely fill the cavity defined below the cover panel 38. High-absorption textiles, such as pique, are to be used as layers 36. Therefore, when the absorbent pad 10 is positioned with respect to the crotch, the liquid gathered in the dam defined by the absorbent pad 10 will either be absorbed by the absorbent layer 24, or will flow downwardly towards the layers 36 guided by the trough-like shape of the concavity 26. The absorbent pad 10 may be provided with different quantities of layers 36 in accordance with various degrees of incontinence problems.

Referring to FIG. 5, the fasteners 32 and 34, herein illustrated as being front and rear loops, are provided for engaging with hooks to be provided on an undergarment, thereby enabling the absorbent pad 10 to be secured to the undergarment. In FIG. 6, the hooks are illustrated at 33 and 35 in the undergarment U. The shape of the absorbent pad 10 allows it to be secured to any type of undergarment. Other typical fastening configurations include Velcro®, sticky surfaces or the like.

There are many various ways to manufacture the absorbent pad 10. The following sequence of steps is described for indicative purposes, and does not limit the manufacturing of the absorbent pad 10 thereto. The resilient core 14 is a result of typical molding or foam-cutting processes. The resilient core 14 is already shaped as en elongated pear when the impermeable outer plastified layer 16 is to be merged thereto. In order to merge the outer plastified layer 16 onto the resilient core 14, two sheets of plastified material sandwich the resilient core 14 therebetween, and an inner joint line 20 and the outer joint line 22 result from ultrasound bonding of the plastified material together so as to completely surround the resilient core 14. The bottom panel 18 is fastened to the contour rib 12 in a similar fashion. It is pointed out that, prior to merging the bottom panel 18 to the contour rib 12, the absorbent layer 24 may be sandwiched between the contour rib 12 and the bottom panel 18. On the other hand, the absorbent layer 24 may be squeezed between the contour rib 12 and the absorbent layer 24 once these two have been merged together. The cover panel 38 is welded to the absorbent pad 10 in a similar fashion.

It is within the ambit of the present invention to cover any obvious modifications of the embodiments described herein, provided such modifications fall within the scope of the appended claims.

1. An absorbent pad for receiving therein at least one of urine and feces, said absorbent pad comprising an elongated body having a bottom panel with a peripheral wall projecting from a periphery of said bottom panel to define a concavity, a layer of absorbent material in said concavity, said bottom panel and said peripheral wall having impermeable outer surfaces such that liquid received in said concavity is retained therein and is absorbed by said layer of absorbent material, said absorbent pad adapted for being positioned between legs of a person such that said concavity faces a crotch of the person for receiving at least one of urine and feces in said concavity, said peripheral wall being elevated so as to prevent said absorbent layer in said concavity of said absorbent pad from coming into contact with skin of the person.

2. The absorbent pad according to claim 1, further comprising at least one handle tab secured at one of a front and rear end thereof for manipulating said absorbent pad, said handle tab enabling washing of said absorbent pad so as to remove urine and feces therefrom for reusing said absorbent pad.

3. The absorbent pad according to claim 2, further comprising handle tabs secured at a front and at a rear end thereof for manipulating said absorbent pad, said handle tabs enabling wringing of said absorbent pad so as to remove liquid therefrom for reusing said absorbent pad.

4. The absorbent pad according to claim 1, wherein said elongated body as an elongated-pear shape, with a larger end of said cavity being positioned in the buttock area of the person.

5. The absorbent pad according to claim 4, wherein a portion of said concavity in said larger end of said absorbent pad is covered with an impermeable panel and is filled with absorbent material, for increasing an absorption capacity of said absorbent pad when said absorbent pad is used solely for receiving urine therein, said impermeable panel preventing said absorbent material from coming into contact with skin of the person.

6. The absorbent pad according to claim 1, wherein an underface of said bottom panel has fastening members on front and rear ends of said absorbent pad, for being secured to corresponding fastening members in undergarment of the person.

7. The absorbent pad according to claim 1, wherein said peripheral wall is a contour rib having a circular cross-section, said contour rib consisting of a resilient core with an outer plastified layer, said outer plastified layer being impermeable such that said resilient core does not come into contact with liquid received in said absorbent pad.

8. The absorbent pad according to claim 1, wherein said handle tabs are loops.

9. A method for washing an absorbent pad having absorbed liquid therein, comprising the steps of:
i) providing handle tabs at opposed ends of said absorbent pad;
ii) holding said absorbent pad by said handle tabs; and
iii) removing liquid absorbed in said absorbent pad by twisting said tabs in opposed directions to wring said absorbent pad.

9. The method according to claim 8, further comprising, between the steps ii) and iii), the step of rinsing said absorbent pad in water.

10. An absorbent pad in combination with an undergarment,
said absorbent pad being adapted for receiving therein at least one of urine and feces, said absorbent pad comprising an elongated body having a bottom panel with a peripheral wall projecting from a periphery of said bottom panel to define a concavity, a layer of absorbent material in said concavity, said bottom panel and said peripheral wall being impermeable such that liquid received in said concavity is retained therein and is absorbed by said layer of absorbent material, said absorbent pad adapted for being positioned between legs of a person such that said concavity faces a crotch of the person for receiving at least one of urine and feces in said concavity, an underface of said bottom panel having first fastening members on front and rear ends of said absorbent pad; and
second fastening members for corresponding engagement with said first fastening members, said second fastening members adapted for being fixed to said undergarment so as to secure said absorbent pad to the undergarment.
